(19) United States
(12) Patent Application Publication
(10) Pub. No.: US 2005/0153358 A1
(43) Pub. Date: Jul. 14, 2005

(54) High-throughput screening method for determining the enantioselectivity of catalysts, biocatalysts, and agents

(75) Inventors: Manfred T Reetz, Mulheim an der Ruhr (DE); Patrick Tielmann, Sinzheim (DE); Andreas Eipper, Ludwigshafen (DE); Richard Mynott, Mulheim an der Ruhr (DE)

Correspondence Address: Norris McLaughlin & Marcus, 30th Floor, 220 East 42nd Street, New York, NY, 10017, US

(73) Assignee: Studiengesellschaft Kohle mbH, Mulheim an der Ruhr (DE)

(21) Appl. No.: 10/506,144

(22) PCT Filed: Feb. 22, 2003
(86) PCT No.: PCT/EP03/01825

(30) Foreign Application Priority Data
Mar. 1, 2002 (DE) 102 09 177.3

(51) Int. Cl. G01N 33/53; G01N 33/543

(52) U.S. Cl. 435/7.1; 436/518

(57) ABSTRACT

The invention relates to a high-throughput screening method based on NMR spectroscopy for determining the enantioselectivity of reactions which show an asymmetric course. The reactions can be caused by chiral catalysts, agents, or biocatalysts such that said products can be evaluated regarding the enantioselectivity thereof. In one embodiment, isotope-marked pseudo-enantiomers or pseudo-prochiral substrates are used such that the enantioselectivity can be quantified by integrating the NMR signals of the respective substrates and/or products. The use of an automated setup of devices, including microtiter plates, robots, and high-throughput NMR devices, is decisive for the high-throughput process. In a second embodiment of the invention, the automated setup of devices is used to detect in a quantitative manner the products and/or educts that have been derivatized with enantiomer-pure agents in the form of diastereomers. At least 1000 ee determinations can be done per day with accuracy of at least ±5 percent in both embodiments.

The present invention relates to a method for determining the enantioselectivity of kinetic racemate resolutions, and of prochiral compounds reactions which proceed asymmetrically, by using isotope-labeled substrates or using chiral auxiliary reagents, with a high-throughput NMR spectrometer being used as the detection system in a automated measurement process. Consequently, the invention makes it possible to carry out a high-throughput screening of enantioselective catalysts, biocatalysts or agents in a simple manner.

The development of effective methods for generating extensive libraries of enantioselective catalysts using procedures of combinatorial chemistry [review: a) M. T. Reetz, Angew. Chem. 2001, 113, 292-320; Angew. Chem. Int. Ed. 2001, 40, 284-310; b) B. Jandeleit, D. J. Schäfer, T. S. Powers, H. W. Turner, W. H. Weinberg, Angew. Chem. 1999, 111, 2648-2689; c) K. Burgess, H.-J. Lim, A. M. Porte, G. A. Sulikowski, Angew. Chem. 1996, 108, 192-194; Angew. Chem. Int. Ed. Engl. 1996, 35, 220-222; d) B. M. Cole, K. D. Shimizu, C. A. Krueger, J. P. A. Harrity, M. L. Snapper, A. H. Hoveyda, Angew. Chem. 1996, 108, 1776-1779; Angew. Chem. Int. Ed. Engl. 1996, 35, 1668-1671], and for preparing libraries of enantioselective biocatalysts using directed evolution [a) M. T. Reetz, A. Zonta, K. Schimossek, K. Liebeton, K.-E. Jaeger, Angew. Chem. 1997, 109, 2961-2963; Angew. Chem. Int. Ed. 1997, 36, 2830-2832; b) M. T. Reetz, K.-E. Jaeger, Chem.-Eur. J. 2000, 6, 407-412] is a subject of current research. The availability of efficient methods for rapidly screening the enantioselective catalysts or biocatalysts in the respective catalyst libraries is of crucial importance for ensuring the success of these new technologies. In contrast to screening methods for combinatorial active compound chemistry [a) F. Balkenhohl, C. Bussche-Hünnefeld, A. Lansky, C. Zechel, Angew. Chem. 1996, 108, 2436-2488; Angew. Chem. Int. Ed. Engl. 1996, 35, 2288-2337; b) J. S. Früchtel, G. Jung, Angew. Chem. 1996, 108, 19-46; Angew. Chem. Int. Ed. Engl. 1996, 35, 17-42; c) Chem. Rev. 1997, 97(2), 347-510 (issue for combinatorial chemistry); d) G. Jung, Combinatorial Chemistry; Synthesis, Analysis, Screening, Wiley-VCH, Weinheim, 1999], there is a lack of efficient methods for the high-throughput screening of enantioselective catalysts, biocatalysts or optically active agents. While the classical determination of enantiomeric excesses (ee) by means of gas chromatography or liquid chromatography on stationary chiral phases provides a high degree of precision, a disadvantage is that the sample throughput per unit of time is limited. The same applies, in a similar manner, to the conventional NMR-spectroscopic determination of the ee value of an enantiomeric mixture in which the sample (e.g. a chiral alcohol) is firstly reacted, in the laboratory, with an enantiomerically pure derivatizing agent (e.g. α-methoxy-α-trifluoromethylphenylacetyl chloride, “Mosher's acid chloride”) or shift reagent (e.g. 1-(9-anthryl)-2,2,2-trifluoroethanol) followed by NMR spectroscopic analysis of the diastereomeric mixture. It is also very time-consuming to operate such a method.

First assays for solving this type of analytical problem have recently been developed. Thus, a test method which makes it possible to monitor the course of enantioselective hydrolyses of chiral carboxylic esters has, for example, been developed in connection with investigations into the directed evolution of enantio-selective lipases [WO9905288A, Studiengesellschaft Kohle; M. T. Reetz, A. Zonta, K. Schimossek, K. Liebeton, K.-E. Jaeger, Angew. Chem. 1997, 109, 2961-2963; Angew. Chem. Int. Ed. Engl. 1997, 36, 2830-2832]. It is possible to use a photometer assay to monitor enantioselective hydrolyses of lipase variants in microtiter plates. Disadvantages are that precise ee values cannot be obtained and this method is restricted to the chiral carboxylic acid substance class. Similar restrictions apply to a related test method [L. E. Janes, R. J. Kazlauskas, J. Org. Chem. 1997, 62, 45460-45461]. In addition, this restriction applies to methods which are based on pH indicator color changes during an ester hydrolysis [L. E. Janes, A. C. Löwendahl, R. J. Kazlauskas, Chem.-Eur. J. 1998, 4, 2324-2331]. While a method for using DNA microarrays for determining enantiomeric excesses makes it possible to achieve a high sample throughput, the assay involves four steps and is consequently laborious; furthermore, the method is not generally applicable [G. A. Korbel, G. Lalic, M. D. Shair, J. Am. Chem. Soc. 2001, 123, 361-362]. The use, which has recently been introduced, of coupled enzyme reactions for determining enantiomeric excesses (EMDee) has an error range of +/−10% ee, which is too high, and can only be used in certain circumstances [P. Abato, C. T. Seto, J. Am. Chem. Soc. 2001, 123, 9206-9207]. An alternative approach identifying chiral catalysts is based on the mass-spectrometric analysis of isotope-labeled pseudo-enantiomers or pseudo-prochiral substrates [WO 00/58504, Studiengesellschaft Kohle; M. T. Reetz, M. H. Becker, H. W. Klein, D. Stöckigt, Angew. Chem. 1999, 111, 1872-1875; Angew. Chem. Int. Ed. 1999, 38, 1758-1761]. However, the method is restricted to the use of prochiral substrates possessing enantiotopic groups or to kinetic racemate resolutions. A system for screening enantioselective catalysts which is based on parallel capillary electrophoresis has recently been presented [PCT/EP 01/09833, Studiengesellschaft Kohle; M. T. Reetz, K. M. Kühling, A. Deege, H. Hinrichs, D. Belder, Angew. Chem. 2000, 112, 4049-4052; Angew. Chem. Int. Ed. 2000, 39, 3891-3893]. This system made it possible, for the first time, to carry out up to 40000 ee determinations per day. However, the method has thus far only been used for analyzing chiral amines. Another ee screening system is based on enzymic immunoassays [F. Turan, C. Gauchet, B. Mohar, S. Meunier, A. Valleix, P. Y. Renard, C. Créminon, J. Grassi, A. Wagner, C. Miokowski, Angew. Chem. 2002, 114, 132-135; Angew. Chem. Int. Ed. 2002, 41, 124-127]. However, the fact that antibodies directed against the enantiomers have to be cultured in an elaborate process is a disadvantage.

### DESCRIPTION OF THE INVENTION

We have found that the above-described restrictions or disadvantages can be avoided if NMR spectroscopy is used as the detection system, in an automated measurement process, in the method for the high-throughput determination of the enantioselectivity of reactions which are brought about by chiral catalysts or biocatalysts or chiral agents. In a first embodiment of the invention, use is made of isotope-labeled substrates which can be detected by NMR spectroscopy. In addition to monitoring kinetic racemate resolutions and stereoselective reactions of compounds possessing enantiotopic groups, it is also possible to use the present invention to conveniently monitor those enantioselective transformations in which a prochiral compound without enantiotopic groups is converted into a chiral product. It is possible to determine the enantiomeric excess (ee value) by quantifying the NMR signals of the isotope-labeled substrates. In the second embodiment of the invention, enantiomerically pure agents are added, for the derivatization, to the chiral products and/or starting compounds of the reactions to be investigated and the NMR signals of the resulting diastereomers are analyzed quantitatively for determining the ee. Furthermore, the ee can also be determined by using chiral solvents or chiral shift reagents. A throughput of 1000 or more samples per day is possible in both embodiments of the invention.

### DESCRIPTION OF THE FIGURES

FIG. 1: a) Asymmetric transformations of pseudo-enantiomeric (a and b), pseudo-meso (c) and pseudo-prochiral (d) compounds. FG depicts the functional group, while FG′ and/or FG″ symbolize the functional groups which are formed by the reaction; the isotope labeling is identified by an asterisk (*).

FIG. 2: Derivatizing enantiomeric mixtures with chiral auxiliary reagents for the quantification by means of NMR analysis.

FIG. 3: Experimental construction of a high-throughput system for screening for enantioselectivity using NMR and isotope-labeled substrates.

FIG. 4: Experimental construction of a high-throughput system for screening for enantioselectivity using NMR and chiral auxiliary reagents and/or chiral agents for solvents.

FIG. 5: Kinetic racemate resolution of 1-phenylethyl acetate: comparison of the ee determination when using chiral GC and when using high-throughput NMR.

FIG. 6: Methyl signal of the diacetate in the ¹H NMR spectrum using natural ¹³C satellites at a measurement frequency of 300 MHz.

FIG. 7: Methyl signal of the diacetate in the ¹H NMR spectrum using 69% ¹³C labeling ( [symbol] 38% ee) at a measurement frequency of 300 MHz.

FIG. 8: Diastereomer resolution in the ¹H NMR spectrum of the CH group of the ester of racemic phenylethanol using MTPA at a measurement frequency of 300 MHz.

As compared with existing methods, the present invention offers the following advantages:
1) Determination of the ee values of asymmetrically proceeding transformations with an error of at most ±5%, with no restriction in regard to the substance class or the reaction type being made.
2) Determination of the turnover of the reactions being investigated.
3) The screening of reactions in a high-throughput method, with at least 1000 determinations per day being possible.

The detection systems used in the present invention are nuclear resonance spectrometers, in particular those possessing a flow-through cell, which are intended for high-throughput operation [review: a) M. J. Shapiro, J. S. Gounarides, Prog. Nucl. Magn. Reson. Spec. 1999, 35, 153-200; b) C. L. Gavaghan, J. K. Nicholson, S. C. Connor, I. D. Wilson, B. Wright, E. Holmes, Anal. Biochem. 2001, 291, 245-252; c) E. Macnamara, T. Hou, G. Fisher, S. Williams, D. Raftery, Anal. Chim. Acta 1999, 387, 9-16] and have automated sample delivery (use of one or more sample delivery robots or pipetting robots), with one or more measuring cells being used per spectrometer, or several spectrometers being used in parallel, in order to achieve the desired high throughput. Suitable nuclei for this purpose are ¹H, ¹⁹F, ³¹P and ¹³C, with it being possible for the method to be extended to other nucleus types (e.g. ¹¹B, ¹⁵N and ²⁹ Si).

The method can be used for finding or optimizing chiral catalysts, biocatalysts or chiral agents for reactions which proceed asymmetrically. These include:
a) chiral catalysts, chiral agents or biocatalysts such as enzymes, antibodies, ribozymes or phages for the kinetic racemate resolution of compounds such as alcohols, carboxylic acids, carboxylic esters, amines, amides, olefins, alkynes, phosphines, phosphonites, phosphites, phosphates, halides, oxiranes, thiols, sulfides, sulfones, sulfoxides and sulfonamides and their derivatives and combinations;
b) chiral catalysts, chiral agents or biocatalysts for the stereoselective conversion of prochiral compounds, with or without enantiopic groups, with the substrate belonging to the substance classes comprising the carboxylic acids, carboxylic esters, alcohols, amines, amides, olefins, alkynes, phosphines, phosphonites, phosphites, phosphates, halides, oxiranes, thiols, sulfides, sulfones, sulfoxides or sulfonamides (or derivatives and combinations thereof).

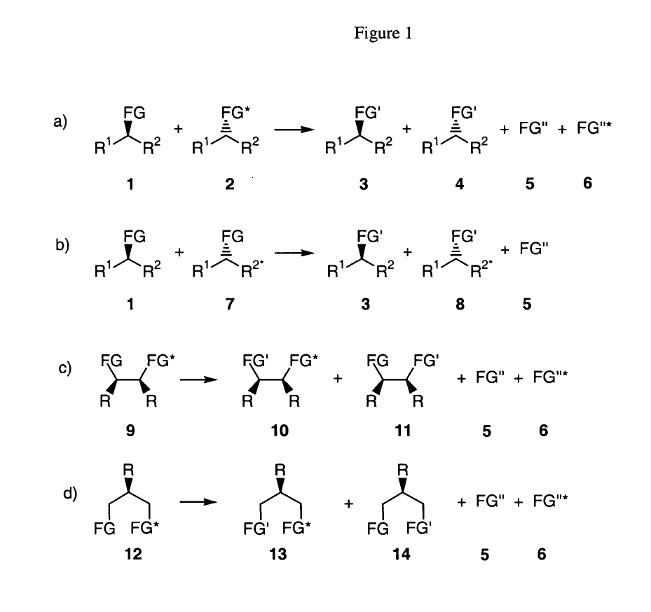

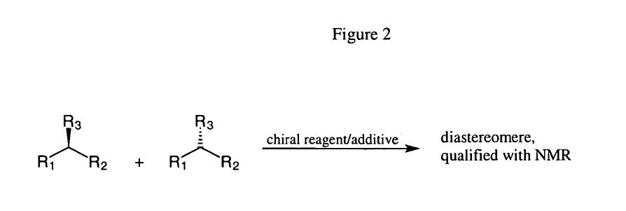

The first embodiment of the invention is based on using isotope-labeled substrates in the form of pseudo-enantiomers or pseudo-prochiral compounds (FIG. 1), with use being made in particular, of ¹³C-labeled substrates. The second embodiment uses chiral auxiliary reagents (FIG. 2).

If one enantomeric form in a conventional racemate is isotope-labeled, such compounds are termed pseudo-enantiomers [cf. M. T. Reetz, M. H. Becker, H.-W. Klein, D. Stöckigt, Angew. Chem. 1999, 111, 1872-1875; Angew. Chem. Int. Ed. 1999, 38, 1758-1761]. If one enantiotopic group of a prochiral substrate is labeled with isotopes, the compound is then termed pseudo-prochiral, for example pseudo-meso. The labels can be introduced in a variety of ways (cf. cases a and b in FIG. 1). In the case of kinetic racemate resolutions of any arbitrary chiral compounds, substrates 1 and 2 or 1 and 7, which differ from each other in their absolute configuration and in the isotope labeling in the functional group FG or in the radical R², are prepared in enantiomerically pure form and mixed in a ratio of 1:1 such that a racemate is simulated (FIG. 1a or b). Following an enantioselective reaction, in which the chemical reaction takes place at the functional group (in the ideal case of a kinetic racemate resolution up to a conversion of 50%), genuine enantiomers 3 and 4, together with unlabeled and labeled achiral byproducts 5 and/or 6, are formed, or else the pseudo-enantiomers 3 and 8 are formed. Pseudo-enantiomers are likewise formed if prochiral compounds are desymmetrised (FIG. 1c or d).

Integrating the corresponding ¹H NMR signals of ¹³C-labeled substrates and/or products, and also of mirror-image, unlabeled substrates and/or products, makes it possible to quantitatively determine the enantio-selectivity (ee value) and the conversion. This is particularly easy to carry out if “isolated” methyl groups have been ¹³C-labeled because the ¹H NMR signal then appears as a doublet whereas the unlabeled methyl group in the enantiomer appears as a singlet. In this way, it is also possible to obtain the selectivity factors (S or E values) in the case of kinetic racemate resolutions [H. B. Kagan, J. C. Fiaud, Top. Stereochem. Vol. 18, Wiley, New York, 1988, 249-330].

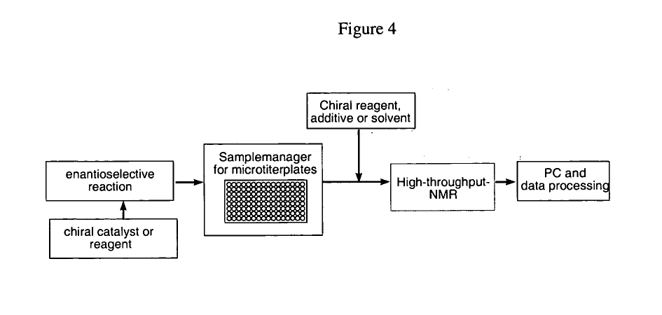

In the second embodiment of the invention, isotope labeling is dispensed with. Instead, the enantiomer mixtures of reactions which proceed asymmetrically are reacted with enantiomerically pure chiral derivatizing agents, NMR shift agents or solvents with the formation of diastereomeric compounds or complexes which are then analyzed by high-throughput NMR spectroscopy (FIG. 4).

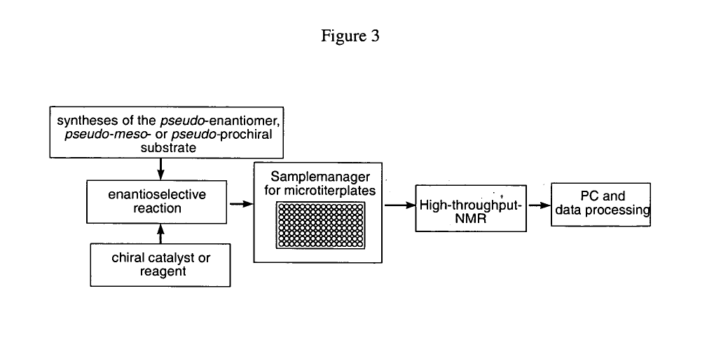

In this second embodiment of the invention (FIG. 2), it is possible to use compounds such as mandelic acid, mandeloyl chloride, O-methylmandelic acid (MPA), O-methylmandeloyl chloride, atrolactic acid, atrolactyl choride, α-methoxy-α-trifluoromethylphenylacetic acid (MTPA, Mosher's acid), α-methoxy-α-trifluoromethyl-phenylacetyl chloride (MTPAC1, Mosher's acid chloride), 2-(9-anthryl)-2-hydroxyacetate (AHA), 9-anthryl-2-methoxyacetate (9-AMA), α-pentafluorophenylpropion-amide, 2-fluorophenylacetic acid (AFPA) or cinchona alkaloid derivatives in enantiomerically pure form as chiral auxiliary reagents. These examples are used for illustrative purposes and do not limit the invention [a) reviews on these and other derivatizing agents: S. K. Latypov, N. F. Galiullina, A. V. Aganov, V. E. Kataev, R. Riguera, Tetrahedron 2001, 57, 2231-2236; b) J. A. Dale, D. L. Dull, H. S. Mosher, J. Org. Chem. 1969, 34, 2543-2549; c) J. A. Dale, H. S. Mosher, J. Am. Chem. Soc. 1973, 95, 512-519]. Chiral NMR shift agents, such as Eu(dcm)₃, where dcm=dicampholyl-methanato, or 1-(9-anthryl)-2,2,2-trifluoroethanol, and also chiral solvents (E. L. Eliel, S. H. Wilen, Stereo-chemistry of Organic Compounds, Wiley, New York, 1994) can likewise be used for forming diastereomeric compounds or complexes. In order to make possible the sought-after high throughput in the two embodiments of the invention, it is necessary to combine automation with miniaturization. Possible instrument set-ups for the two embodiments are shown diagrammatically in FIG. 3 and FIG. 4, respectively.

In this way, it is possible to carry out high-throughput screening of libraries of chiral catalysts, biocatalysts or agents using commercially available microtiter plates and robots (sample managers). After the reaction has taken place, the samples are analyzed by NMR spectroscopy. When the NMR spectrometer is appropriately equipped, it is also possible to employ modern pulse methods, using pulsed field gradients and shaped HF pulses, for the ee determination. When using this combination of commercially available equipment and apparatus parts, it is possible to carry out at least 1000 ee determinations per day with an accuracy of +/−5%.

The assay for the high-throughput screening of an asymmetric reaction using NMR is configured such that, in the case of a kinetic racemate resolution, a pseudo-racemate is first of all prepared from enantiomerically pure isotope-labeled and unlabeled substrate. The racemate resolution is then carried out, for example in 96-well microtiter plates, in the added presence of the catalyst. Finally, the samples are introduced into the flow-through cell of the NMR apparatus using a pipetting and sample dispensing robot (FIG. 3). When chiral derivatizing reagents are used, the procedure is changed in that, after the catalytic reaction has come to an end, the pipetting robot is firstly used to add the reagent to the reaction mixture. It is only after that that the sample is introduced into the flow-through cell (FIG. 4). In both cases, the data sets which are obtained can be automatically analyzed using suitable software, e.g. AMIX® from Bruker.

### EXAMPLE 1

### Kinetic Racemate Resolution of 1-phenylethyl Acetate

The kinetic racemate resolution of 1-phenylethyl acetate by means of hydrolysis, catalyzed by, for example, enzymes such as lipases (wild type or variants), is monitored within the context of a high-throughput assay as shown in FIG. 3, i.e. both enantioselectivity and conversion are determined. 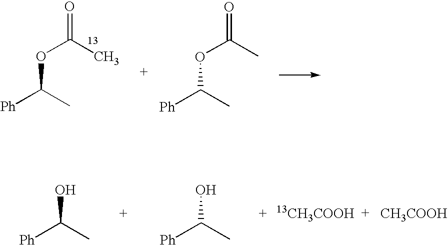
Synthesizing (R)-1-phenylethyl Acetate:

4 ml of pyridine (abs.) and 1.0 g (8.2 mmol) of (R)-1-phenylethanol are dissolved, under argon, in 30 ml of dichloromethane (abs.) in a 50 ml single-necked flask fitted with a tap, and the solution is cooled down to 0° C. 0.97 g (12.3 mmol) of acetyl chloride is then added dropwise, with a white precipitate appearing. The mixture is then stirred overnight at RT and the red solution is quenched with water while cooling with an ice bath. The organic phase is separated off, in each case extracted once with 1M hydrochloric acid and a sat. solution of sodium chloride, and dried over magnesium sulfate. The solvent is separated off on a rotary evaporator and the crude product is subjected to silica gel column chromatography using dichloromethane. Following removal of the solvent in vacuo, and brief drying under high vacuum, 1.24 g (92%) of the desired product are obtained as a clear liquid. Analysis: ¹H NMR (300 MHz, CDCl₃): δ=1.53 (d, ³J_{H.H}=6.6 Hz, 3H); 2.06 (s, 3H); 5.88 (q, ³J_{H.H}=6.6 Hz, 1H); 7.24-7.37 (m, 5H); ¹³C NMR (75.5 MHz, CDCl₃): δ=21.3; 22.2; 72.3; 126.1; 127.9; 128.5; 141.7; 170.3; MS (EI, 70 eV) m/z=164 (M⁺); 122; 104; 77; EA: % C 72.9 (calc. 73.3); % H 7.4 (calc. 7.3).

Synthesizing (S)-1-phenylethyl 2-¹³C-acetate:

4 ml of pyridine (abs.) and 1.0 g (8.2 mmol) of (S)-1-phenylethanol are dissolved, under argon, in 30 ml of dichloromethane (abs.) in a 50 ml single-necked flask fitted with a tap, and the solution is cooled down to 0° C. 0.97 g (12.3 mmol) of 2-¹³C-acetyl chloride is then added dropwise, with a white precipitate appearing. The mixture is then stirred overnight at RT and the red solution is quenched with water while cooling with an ice bath. The organic phase is separated off, in each case extracted once with 1M hydrochloric acid and a sat. solution of sodium chloride, and dried over magnesium sulfate. The solvent is separated off on a rotary evaporator and the crude product is subjected to silica gel column chromatography using dichloromethane. Following removal of the solvent in vacuo, and brief drying under high vacuum, 1.24 g (92%) of the desired product are obtained as a clear liquid. Analysis: ¹H NMR (300 MHz, CDCl₃): δ=1.53 (d, ³J_{H.H}=6.6 Hz, 3H); 2.06 (d, ¹J_{C.H}=129.4 Hz, 3H); 5.88 (q, ³J_{H.H}=6.6 Hz, 1H); 7.24-7.37 (m, 5H); ¹³C NMR (75.5 MHz, CDCl₃): δ=21.3; 22.2; 72.3; 126.1; 127.9; 128.5; 141.7; 170.7; MS (EI, 70 eV): m/z=165 (M⁺); 122; 104; 77; 44; EA: % C 72.6 (calc. 73.3); % H 7.5 (calc. 7.3).

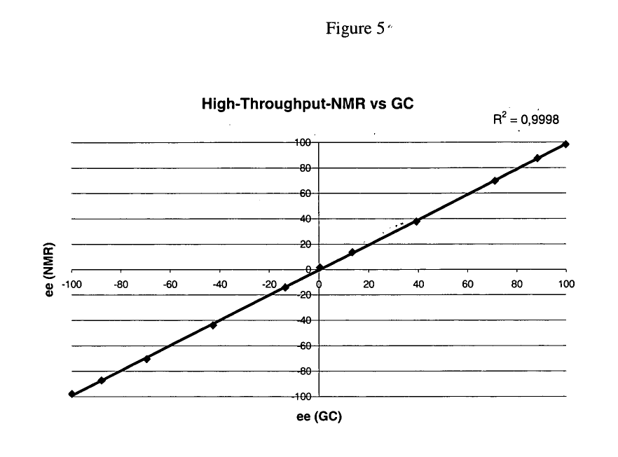

In preliminary experiments, the pseudo-enantiomers were mixed in various ratios. The mixtures which were obtained in this connection were initially investigated by means of gas chromatography on a chiral stationary phase in order to determine the pseudo-ee values. The same samples were then investigated by NMR spectroscopy. Comparison of the two data sets shows agreement within a limit of +/−2% (Table 1) and a high correlation (R²=0.9998 in FIG. 5).

TABLE 1
Mixtures of 35 μl to 700 μl of CDCl₃.

| | ee (%) | ee (%) |
|---|---|---|
| Batch | by GC | by ¹H NMR |
| 1 | 100 (S) | 98.2 (S) |
| 2 | 88.5 (S) | 87.4 (S) |
| 3 | 71.2 (S) | 69.6 (S) |
| 4 | 39.2 (S) | 37.8 (S) |
| 5 | 13.4 (S) | 13.6 (S) |
| 6 | 0.4 (S) | 1.6 (S) |
| 7 | 13.6 (R) | 14.2 (R) |
| 8 | 42.8 (R) | 44.0 (R) |
| 9 | 69.6 (R) | 70.6 (R) |
| 10 | 87.8 (R) | 87.2 (R) |
| 11 | 100 (R) | 98.0 (R) |

In order to achieve a sample throughput which is as high as possible, the measurement method can be reduced to a cycle time of approximately one minute. This does not impair the precision of the analysis; backmixing with the previous sample remains less than 1%. Typical results are summarized in Table 2.

TABLE 2
Mixtures of 1.3 to 1.7 mg per 1 ml of CDCl₃ in
the high-throughput NMR method (approx. 1 min per cycle).

| | ee (%) | ee (%) |
|---|---|---|
| Batch | by GC | by ¹H NMR |
| 1 | 39.2 (S) | 38.5 (S) |
| 2 | 39.2 (S) | 38.2 (S) |
| 3 | 39.2 (S) | 38.3 (S) |
| 4 | 13.6 (R) | 12.7 (R) |
| 5 | 13.6 (R) | 12.2 (R) |
| 6 | 13.6 (R) | 12.8 (R) |
| 7 | 42.8 (R) | 41.9 (R) |
| 8 | 42.8 (R) | 41.1 (R) |
| 9 | 42.8 (R) | 41.8 (R) |

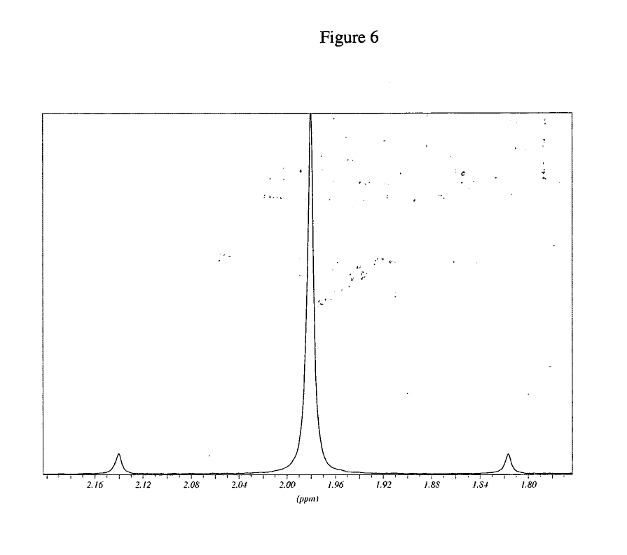

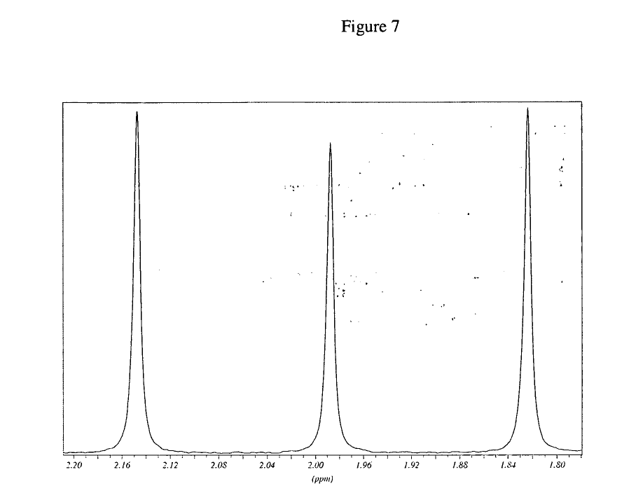

The ratios of the methyl signals in the ¹H NMR spectrum (FIGS. 6 and 7) were analyzed automatically using the Bruker AMIX® software.

### EXAMPLE 2

### Kinetic Racemate Resolution of Methyl 2-phenylpropionate

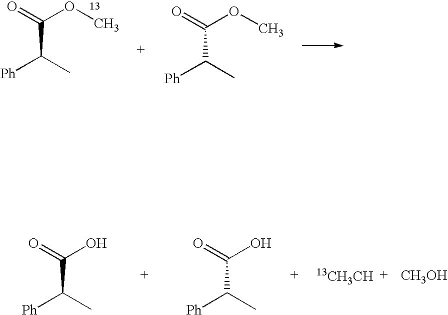 Synthesizing Methyl (R)-2-phenylpropionate:

600 mg (4.0 mmol) of (R)-2-phenylpropionic acid and 912 mg (6.0 mmol) of cesium fluoride are taken up in 12 ml of dimethylformamide (abs.) in a 25 ml single-necked flask fitted with a tap, and the solution is cooled down to 13±1° C. using a cryostat. 1.93 g (13.6 mmol) of methyl iodide are then added and the mixture is stirred at this temperature for 46 h. After that, a little ethyl acetate is added and removed in vacuo together with the excess methyl iodide. The residue is taken up in ethyl acetate and this solution is extracted once with a sat. solution of sodium hydrogen carbonate and dried over magnesium sulfate. After the solvent has been removed on a rotary evaporator, the crude product is subjected to silica gel column chromatography using hexane/ethyl acetate 8:2. Following removal of the solvent in vacuo, and brief drying under high vacuum, 454 mg (69%) of the product are obtained as a clear liquid. Analysis: ¹H NMR (300 MHz, CDCl₃): δ=1.50 (d, ³J_{H.H}=7.2 Hz, 3H); 3.65 (s, 3H); 3.72 (q, ³J_{H.H}=7.2 Hz, 1H); 7.23-7.35 (m, 5H); ¹³C NMR (75.5 MHz, CDCl₃): δ=18.6; 45.4; 52.0; 127.1; 127.5; 128.6; 140.6; 175.0; MS (EI, 70 eV): m/z=164 (M⁺); 105; 77; 51; EA: % C=73.2 (calc. 73.3); % H 7.5 (calc. 7.3).

Synthesizing ¹³C-methyl (S)-2-phenylpropionate:

600 mg (4.0 mmol) of (S)-2-phenylpropionic acid and 912 mg (6.0 mmol) of cesium fluoride are taken up in 12 ml of dimethylformamide (abs.) in a 25 ml single-necked flask fitted with a tap and this solution is cooled down to 13±1° C. using a cryostat. 1.93 g (13.6 mmol) of ¹³C-methyl iodide are then added and the mixture is stirred at this temperature for 46 h. After that, a little ethyl acetate is added and removed in vacuo together with the excess methyl iodide. The residue is taken up in ethyl acetate and this solution is extracted once with a sat. solution of sodium hydrogen carbonate and dried over magnesium sulfate. After the solvent has been removed on a rotary evaporator, the crude product is subjected to silica gel column chromatography using hexane/ethyl acetate 8:2. Following removal of the solvent in vacuo, and brief drying under high vacuum, 454 mg (69%) of the product are obtained as a clear liquid. Analysis: ¹H NMR (300 MHz, CDCl₃): δ=1.50 (d, ³J_{H.H}=7.2 Hz, 3H); 3.65 (d, ³J_{C.H}=146.9 Hz, 3H); 3.71 (q, ³J_{H.H}=7.1 Hz, 3H); 7.22-7.35 (m, 5H); ¹³C NMR (75.5 MHz, CDCl₃): δ=18.6; 45.4; 52.0; 127.1; 127.5; 128.6; 140.6; 175.0; MS (EI, 70 eV): m/z=165 (M⁺); 105; 77; 51; EA: % C 72.8 (calc. 73.3); % H 7.4 (calc. 7.3).

In order to evaluate the screening system, the corresponding esters were mixed in various ratios and determined both by means of GC and by means of high-throughput NMR; the results are summarized in Table 3. In all cases, the error is ≦2% ee.

TABLE 3
Mixtures of 10 μl per 700 μl of CDCl₃.

| | ee (%) | ee (%) |
|---|---|---|
| Batch | by GC | by ¹H NMR |
| 1 | 100 (S) | 98.2 (S) |
| 2 | 82.6 (S) | 82.8 (S) |
| 3 | 76.4 (S) | 77.0 (S) |
| 4 | 58.0 (S) | 58.8 (S) |
| 5 | 29.8 (S) | 30.4 (S) |
| 6 | 0 | 0.6 (R) |
| 7 | 31.0 (R) | 29.0 (R) |
| 8 | 58.4 (R) | 57.2 (R) |
| 9 | 74.6 (R) | 74.0 (R) |
| 10 | 81.2 (R) | 81.4 (R) |
| 11 | 100 (R) | 98.2 (R) |

The ratios of the methyl signals (FIGS. 6 and 7) in the ¹H NMR spectrum were analyzed automatically using the Bruker AMIX® software.

### EXAMPLE 3

### Enantioselective Hydrolysis of meso-1,4-diacetoxy-2-cyclopentene

This examples relates to the reaction of a pseudo-prochiral compound which carries enantiotopic groups (in this case acetoxy groups). 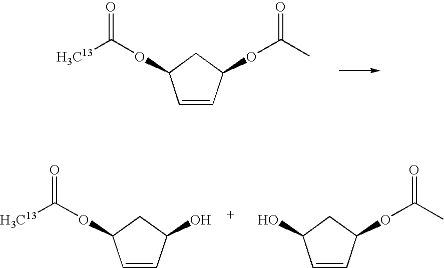
Synthesizing (1S,4R)-cis-1-(2-¹³C-acetoxy)-4-acetoxy-2-cylcopentene:

5.00 mg (35.2 mmol) of (1S,4R)-cis-4-acetoxy-2-cyclopenten-1-ol, 4.27 ml (4.18 g, 6.95 mmol) of pyridine and 100 ml of dichloromethane are initially introduced, while excluding air and moisture, into a 250 ml nitrogen flask and this mixture is cooled down to 0° C. While stirring, 3.00 ml (3.44 g, 42.4 mmol) of 2-¹³C-acetyl chloride are added dropwise within the space of 10 min. The mixture is warmed to room temperature within the space of 12 h and extracted consecutively in each case twice with 50 ml of 1 M hydrochloric acid solution, a saturated solution of sodium hydrogen carbonate and a saturated solution of sodium chloride. The organic phase is dried over magnesium sulfate, separated off from the drying agent by filtration and freed of the solvent on a rotary evaporator. The crude product is loaded onto silica gel and purified chromatographically using hexane/ethyl acetate 5:1. The product fractions are combined and freed of the solvents on a rotary evaporator. Following drying under a oil pump vacuum, a clear liquid remains (6.38 h, 97%). Analysis: ¹H NMR (CDCl₃, 300 MHz): δ=1.71-1.78 (m, 2H); 2.07 (s, 3H); 2.07 (d, ¹J_{C.H}=130 Hz, 3H); 2.83-2.93 (m, 2H); 5.55 (dd, ³J_{H.H}=3.8 Hz, ²J_{H.H}=7.5 Hz, 2H); 6.10 (s, 2H); ¹³C NMR (CDCl₃, 75 MHz): δ=21.5; 37.5; 76.9; 135.0; 171.1; MS (EI, 70 eV): m/z=183 (M⁺); 82; 54; 46; 43; EA: C, 57.8% (calc. 57.7%); H, 6.5% (calc. 6.5%).

In order to evaluate the screening system, the corresponding monoacetates were mixed in various ratios and determined both by GC and by high-throughput NMR. The results are summarized in Table 4.

TABLE 4
Mixtures of 1 mg per 1 ml of CDCl₃.

| | ee (%) | ee (%) |
|---|---|---|
| Batch | by GC | by ¹H NMR |
| 1 | 100 (S) | 99.5 (S) |
| 2 | 82.4 (S) | 82.6 (S) |
| 3 | 63.0 (S) | 63.8 (S) |
| 4 | 43.0 (S) | 44.3 (S) |
| 5 | 6.4 (S) | 9.2 (S) |
| 6 | 2.6 (S) | 3.6 (S) |
| 7 | 19.6 (R) | 17.3 (R) |
| 8 | 41.6 (R) | 38.3 (R) |
| 9 | 64.4 (R) | 63.9 (R) |
| 10 | 82.2 (R) | 81.8 (R) |
| 11 | 99.9 (R) | 97.5 (R) |

The ratios of the methyl signals in the ¹H NMR spectrum (FIGS. 6 and 7) were analyzed automatically using the Bruker AMIX® software.

### EXAMPLE 4

### Kinetic Racemate Resolution of 2-butanol

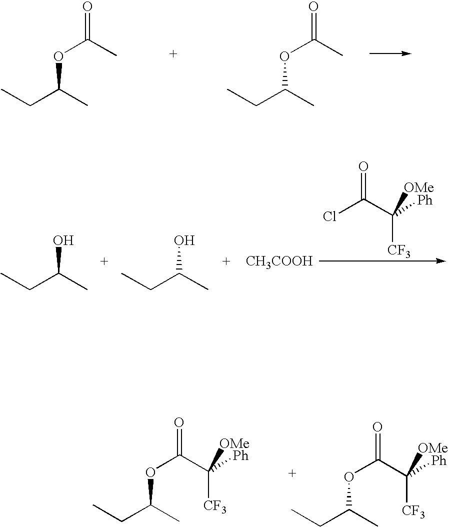

The alcohol was first of all derivatized with Mosher's acid cloride in order to prepare the corresponding diastereomeric esters. After that, the samples were tested in a high-throughput NMR apparatus and the ee values were calculated by automatically integrating the CH₂ signals of the diastereomers in the ¹H NMR spectrum. As a control, the enantiomeric purity of the same samples was determined by gas chromatography. The ee values which were determined by means of high-throughput NMR and GC are compared with each other in Table 5.

TABLE 5
Mixtures of 1 mg per 1 ml of CDCl₃

| | ee (%) | ee (%) |
|---|---|---|
| Batch | by GC | by ¹H NMR |
| 1 | 100 (S) | 100 (S) |
| 2 | 68.4 (S) | 70.9 (S) |
| 3 | 47.6 (S) | 52.7 (S) |
| 4 | 36 (S) | 34.2 (S) |
| 5 | 19 (S) | 17.6 (S) |
| 6 | 2.2 (R) | 3.4 (R) |
| 7 | 10.4 (R) | 12.3 (R) |
| 8 | 35 (R) | 40.5 (R) |
| 9 | 49.8 (R) | 56 (R) |
| 10 | 66.4 (R) | 66.2 (R) |
| 11 | 100 (R) | 100 (R) |

The ratios of the CH₂ signals of the diastereomers were analyzed automatically using the Bruker AMIX® software.

### EXAMPLE 5

### Kinetic Racemate Resolution of 1-phenylethanol

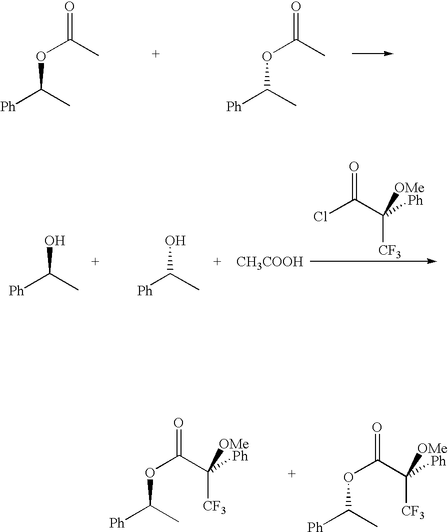

The alcohol was first of all derivatized with Mosher's acid chloride in analogy with Example 4 in order to prepare the corresponding diastereomeric esters. After that, the samples were tested in a high-throughput NMR apparatus and the ee values were calculated by automatically integrating the CH signals of the diastereomers in the ¹H NMR spectrum. As a control, the enantiomeric purity of the same samples was determined by gas chromatography. The ee values which were determined using the high-throughput NMR apparatus and by means of GC are compared in Table 6.

TABLE 6
Mixtures of 1 mg in 1 ml of CDCl₃

| | ee (%) | ee (%) |
|---|---|---|
| Batch | by GC | by ¹H NMR |
| 1 | 100 (S) | 100 (S) |
| 2 | 82.7 (S) | 86.0 (S) |
| 3 | 65.0 (S) | 66.7 (S) |
| 4 | 47.7 (S) | 55.0 (S) |
| 5 | 35.4 (S) | 38.7 (S) |
| 6 | 11.4 (S) | 16.3 (S) |
| 7 | 6.6 (R) | 3.5 (R) |
| 8 | 25.2 (R) | 21.9 (R) |
| 9 | 49.6 (R) | 45.9 (R) |
| 10 | 74.8 (R) | 75.4 (R) |
| 11 | 100 (R) | 100 (R) |

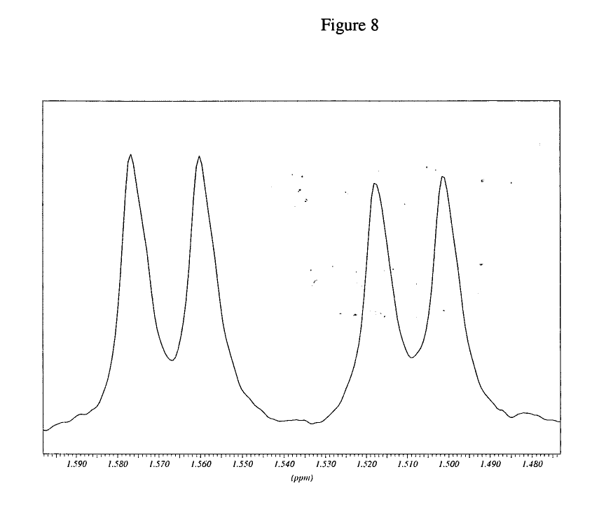

The ratios of the CH signals of the diastereomers (FIG. 8) were analyzed automatically using the Bruker AMIX® software.

1. A method for high-throughput determination of the enantioselectivity of reactions which are brought about by chiral catalysts, biocatalysts or chiral agents, characterized in that nuclear magnetic resonance (NMR) spectroscopy is used as the detection system in an automated measuring process.

2. The method as claimed in claim 1, characterized in that suitable isotope-labeled substrates are used for the NMR detection.

3. The method as claimed in claim 2, wherein the isotope-labeled substrates are pseudo-enantiomers.

4. The method as claimed in claim 2, wherein the isotope-labeled substrates are pseudo-prochiral compounds possessing enantiotopic groups.

5. The method as claimed in claims 2-4, wherein the ratio of enantiomeric products and/or starting compounds is determined quantitatively by means of the NMR-spectroscopic integration of the signals of isotope-labeled and unlabeled compounds.

6. The method as claimed in claims 2-5, wherein the isotope labeling is performed using ¹³C or D.

7. The method as claimed in claims 1-5, wherein the NMR-active nuclei employed are ¹H, ¹³C, ³¹P or ¹⁹F.

8. The method as claimed in claim 1, characterized in that enantiomerically pure agents and/or chiral solvents or chiral shift reagents are added to the chiral products and/or starting compounds of the reactions and the NMR signals of the diastereomers are measured.

9. The method as claimed in claims 1-8, wherein a high-throughput NMR apparatus is used as the detection system.

10. The method as claimed in claim 9, wherein a sample dispensing robot is used together with the high-throughput NMR apparatus.

11. The method as claimed in claims 1-10, wherein one or more sample dispensing robots, one or more microtiter plates, one or more NMR spectrometers and one or more measuring cells are used in the automated measuring process.

12. The method as claimed in claims 1-11, wherein at least 1000 ee determinations per day are possible.
